(19) United States
(12) Patent Application Publication
(10) Pub. No.: US 2018/0071134 A1
(43) Pub. Date: Mar. 15, 2018

(54) DEVICE FOR TREATMENT OF SLEEP RELATED DISORDERS, A PROCESS TO MAKE THE DEVICE AND A PROCESS TO USE THE DEVICE

(71) Applicant: GORDON HONIG, Wilmington, DE (US)

(72) Inventor: GORDON HONIG, Wilmington, DE (US)

(21) Appl. No.: 15/364,943

(22) Filed: Nov. 30, 2016

Related U.S. Application Data
(60) Provisional application No. 62/394,251, filed on Sep. 14, 2016.

(51) Int. Cl. A61F 5/56

(52) U.S. Cl. CPC A61F 5/566

(57) ABSTRACT

A device for positioning the teeth for the treatment of sleeping disorders containing an upper receptor capable of covering a partial amount of the user's upper teeth and lower receptor capable of covering a partial amount of the user's lower teeth and at least two connecting structures to connect the upper receptor to the lower receptor whereby a slot is formed between the upper receptor and the lower receptor to allow for breathing. The invention is related to a process to make the device and the method of using the device.

### RELATED APPLICATIONS

This application claims benefit to U.S. Provisional Application 62/394,251 filed Sep. 14, 2016 which is incorporated by reference for all useful purposes.

The present invention relates to a device for the treatment of sleep related disorders, including snoring and sleep apnea and a process to make and use the device.

### BACKGROUND OF THE INVENTION

Normally, the muscles of the upper part of the throat keep the airway open to permit air flow into the lungs. When the muscles of the soft palate at the base of the tongue and the uvula relax and sag, the relaxed tissues may vibrate as air flows past the tissues during breathing, resulting in snoring.

In more serious cases, the airway becomes blocked, making breathing labored and noisy, or even stopping it altogether. Apnea appears when the upper airway passages are being sucked close to the rear part of the throat when the person is trying to breathe during sleep.

There are several methods and devices presently available for the treatment of snoring and obstructive sleep apnea (OSA). One device is the CPAP (Continuous Positive Airway Pressure) machines. They entail wearing of a mask, headgear, and flexible hose which is attached to the air pump. Although effective, the CPAP machine is not widely accepted by the patients. Discomfort, the sound of the air pump, claustrophobia and the stigma of being seen while wearing the mask, headgear, and hose have all been listed as reasons for not continuing use of the CPAP.

There are oral appliances or devices which are designed to displace the mandible (lower jaw) or tongue in an anterior (forward) direction. The purpose is to displace the tongue in an anterior direction, to prevent the collapse of the tongue and soft tissues in the back of the throat so that the airway stays open during sleep. These devices have been only partially successful and are not tolerated by a significant percentage of the patients who have them fitted.

Currently, different types of devices are available to specially trained dentists to treat sleep disordered breathing. At first glance, this number appears overwhelming but on close examination each of the appliances falls basically into one of two categories and the diverse variety is simply a variation of a few major themes, mandibular repositioning appliances (MRA) and tongue retaining devices (TRD).

Mandibular repositioning appliances are by far the most numerous types on the market. They all function to reposition and maintain the lower jaw (mandible) in a protruded position during sleep. It is felt that this serves to open the airway by indirectly pulling the tongue forward since the tongue is attached to the lower jaw, by stimulating activity of the muscles in the tongue and making it more rigid, and by holding the lower jaw and other structures in a stable position to prevent opening of the mouth.

The second type of device as discussed above is a TRD. TRDs comprise two fused splints with an elastic sleeve or bulb sandwiched between them. The patient inserts his or her tongue into the sleeve, which holds the tongue in the forward position. When the tongue is in a forward position, the back of the tongue does not collapse during sleep and obstruct the airway in the throat.

U.S. Publication No. 2010/0311008, U.S. Publication No 2007/0163603 and U.S. Pat. No. 6,408,851 B1 relate to a device for positioning the tongue for the treatment of disorders including snoring and obstructive OSA and OSA Syndrome.

U.S. Pat. No. 9,408,743 relates to an apparatus for reducing obstructive sleep apnea, snoring and/or nasal drainage. The upper member includes molar extensions projecting away from the upper member and toward the member molar regions so that when the user bites or clenches, the upper right and lower right extensions impinge on one another in substantially overlapping fashion, as do the upper left and lower left extensions. The upper and lower members have an anterior shape to form a gap sufficient for the user's tongue to extend into the gap, and each molar extension may include a magnet.

### SUMMARY OF THE INVENTION

While the prior art devices represent attempts to solve sleeping disorder problems, they are rather uncomfortable for the wearer, and they do not appear convincing with respect to their capability of achieving an effective and anti-OSA syndrome effect.

Thus, the need remains for a simple device for reducing or preventing snoring and/or obstructive sleep apnea, without the disadvantages of current devices and mandibular repositioning appliances. The present invention provides such a device, a process to make the device and a process to use the device.

One of object of the invention is a device for positioning the teeth for the treatment of sleeping disorders comprising an upper receptor capable of covering a partial amount of the user's upper teeth and lower receptor capable of covering a partial amount of the user's lower teeth and at least two connecting structures to connect the upper receptor to the lower receptor whereby a slot if formed between said upper receptor and said lower receptor to allow for breathing wherein said upper receptor and said lower receptor are generally aligned with each other to generally align the upper teeth with the lower teeth.

Another object of the invention is a treatment for sleep disorders which comprises a user inserting the inventive device in the user's mouth prior to sleeping.

Another object of the invention is a treatment for sleep disorders which comprises a user inserting the device produced according to the inventive method in the user's mouth prior to sleeping.

Another object of the invention is method to make the device of this invention, which comprises

making an exact replica models of the patient's dentition and creating a model, wherein the model extends at most from the second bicuspid tooth to the second bicuspid tooth for the upper teeth and extends at most from the left second bicuspid tooth to the right second bicuspid tooth on the lower teeth,
thermoforming a plastic on the model to form an upper and lower receptor,
cooling the plastic,
removing the plastic from the model,
optionally trimming that the lower and upper receptor and
bonding the two receptors together in at least two locations and thereby forming a slot in front between the two receptors.

### A BRIEF DESCRIPTION OF THE FIGURES

FIG. 1 illustrates an isometric view of the device according to the invention.

FIG. 2 illustrates a front view of the device according to the invention.

FIG. 3 illustrates a side view of the device according to the invention inserted on the patient's teeth or exact replica models of the patient's dentition.

FIG. 4 illustrates a side view of the patient's teeth or dentition before the insertion of the device of the invention.

FIG. 5 illustrates a side view of the patient's teeth or dentition after the insertion of the device of the invention.

FIG. 6 illustrates an enlarged view of the portion of FIG. 4 labelled FIG. 6.

FIG. 7 illustrates an enlarged view of the portion of FIG. 5 labelled FIG. 7.

FIG. 8-10 illustrates the device according to the invention.

### A DETAILED DESCRIPTION OF THE INVENTION

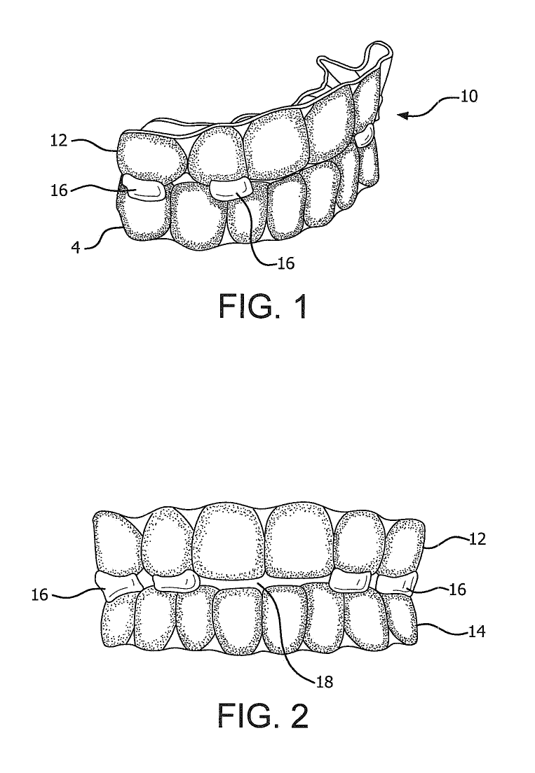

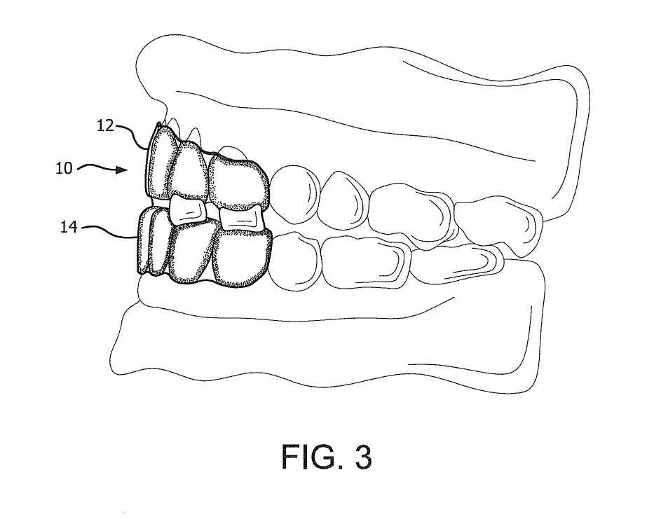

The invention is drawn to a device 10 for positioning the teeth for the treatment of sleeping disorders comprising an upper receptor 12 capable of covering a partial amount of the user's upper teeth and lower receptor 14 capable of covering a partial amount of the user's lower teeth and at least two connecting structures 16 to connect the upper receptor 12 to the lower receptor 14 whereby a slot 18 is formed between said upper receptor 12 and said lower receptor 14 to allow for breathing and said lower receptor are generally aligned with each other to generally align the upper teeth with the lower teeth (see FIGS. 1 and 2).

Throughout the application the term device and appliance are used interchangeably.

The upper receptor preferably extends from the second bicuspid tooth to the second bicuspid tooth and said lower receptor covers the left second bicuspid tooth to the right second bicuspid tooth.

The upper receptor most preferably extends from the right canine tooth to the left canine tooth and said lower receptor most preferably extends from the left first bicuspid to the right first bicuspid tooth.

Sleeping disorders include snoring, OSA, OSA Syndrome and/or nasal drainage.

Fabrication of the Inventive Device

Exact replica models of the patient's dentition are fabricated from either analog impression or digital scanning. These are well known technologies.

There are basically two types of scanners on the market today. There are systems that use blue LED (light-emitting diode). These systems are optical scanners that depend upon a reflective surface and require a contrasting medium or powder to acquire a representation of the tooth morphology. There are also systems that use laser technology to scan and measure distances from the tooth surface to acquire the image. They do not require powder.

There are five major systems on the market today—Cadent iTero, 3M ESPE Lava COS, CEREC by Sirona, E4D by D4D Technologies and a Carestream Dental CBCT system.

The Cadent and 3M ESPE machines produce models that are sent to the lab to produce the restoration of choice. Sirona and D4D include milling units for immediate fabrication of the final restoration chairside.

The iTero is an optically assisted laser scanner that does not require powder. The optics allow for real-time imaging. This allows seeing the model being built as you proceed through the scanning process. A milled model is produced, allowing for fabrication of restorative needs.

Lava COS (chairside oral scanner) is a video-type format that uses the blue light emitting diode (LED) scanning system that requires powder. The downloaded scan information produces a stereo lithography (SLA) model that allows for fabrication of any type of restoration.

The CEREC AC by Sirona can be used for fabrication of a model-only system. It uses a blue LED optical scanner and requires the use of powder.

E4D's system uses a laser scanner and does not require powder. E4D offers what it refers to as TSS or three sources scanning. This allows the dentist to directly scan intraorally, take a conventional impression, and scan the impression or pour the impression, and then scan the model.

Using a Carestream Dental CBCT system, practitioners can scan conventional impressions to create high-precision 3D models quickly and easily. This innovative technique on the market digitizes impressions so teeth can be designed and milled.

There are two types of modeling systems are used, which are SLA and milling. SLA is a rapid prototyping technology that produces an accurate model.

Milled models are produced using a plastic such as a polymer that is placed in a milling machine to remove excess material to accuracies within about 20 microns. SLA produces models for CEREC and Lava COS, while Cadent uses milling to produce its model.

Using a Carestream Dental CBCT system, practitioners can scan conventional impressions to create high-precision 3D models quickly and easily. This innovative technique on the market digitizes impressions so crowns can be designed and milled.

From the models, a plastic is thermoformed on the models. After cooling, they are removed from the models and trimmed so that the lower (mandibular) appliance extends preferably from the left first bicuspid to the right first bicuspid.

The plastic material used in the construction of the upper and lower receptors 12 and 14 may comprise any moldable plastic (polymeric) material that is approved for use in oral medical devices and appliances for human use and that may be custom fitted for each user and tooth-retained via friction grip. The material may comprise a single material, or combination of materials. The material can be a flat sheet and may comprise more than one layer of material, and each layer may be the same or different. The polymeric materials may be filled with various fillers, extenders, pigments, and other additives. In embodiments consisting essentially of moldable, biocompatible polymeric material, these fillers, extenders, pigments, and other additives are present in limited amounts to the extent necessary to substantially exceed minimum safety and effectiveness standards. Suitable polymeric materials include thermoplastics, thermosetting polymers, elastomers, and thermoplastic elastomers. The polymeric materials may comprise co-polymers, ter-polymers, and blends of two or more chemical types of polymers, or blends of two or more polymers of the same chemical type, for example, a blend of two thermoplastics having different molecular weights.

Examples of specific polymers include polyurethanes, polyacrylics, polyvinyls, polyvinyl alcohols, and the like. An example of a suitable polymeric material is a durable fade-proof acrylic that retains its shape and color for at least four-five years. The material used for the upper and lower receptor is preferably a plastic material such as but not limited to Essix C+ plastic, Essix® PLUS™ Plastic, Essix ACE® Plastic, Dreve Drufosoft® Pro, Dreve Kombiplast, Dreve Biolon, Essix® Nightguard Laminate, Essix® Dual Laminate, Essix® Bleach Tray and Model Duplication Material, Essix A+® Plastic or Essix Tray Rite® Plastic (Essix is a registered trademark of Raintree Essix). The thickness is preferably about 1 mm thick. If the material is too thick it will not be as comfortable to the user.

The upper (maxillary) receptor 12 is preferably trimmed and extends preferably from the right canine tooth to the left canine tooth. It is possible to extend it over additional teeth.

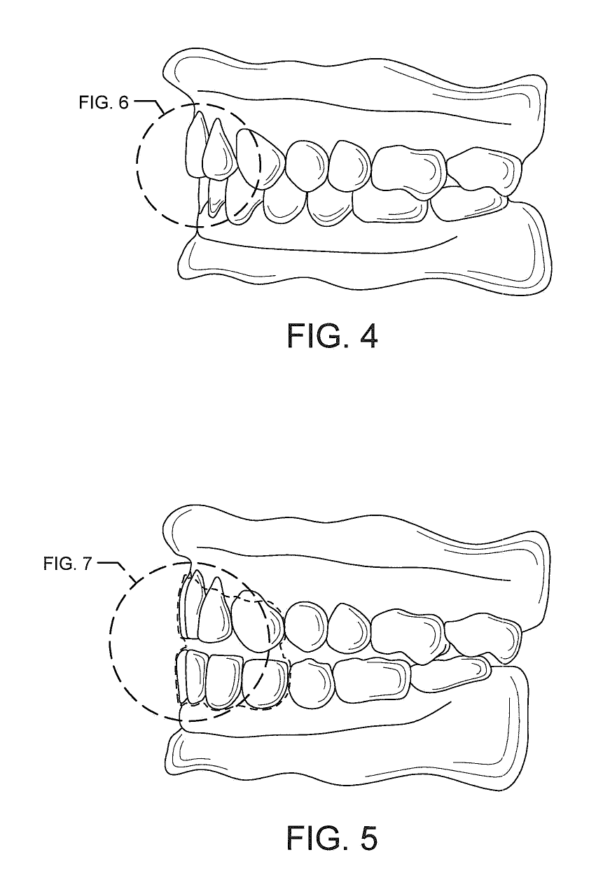

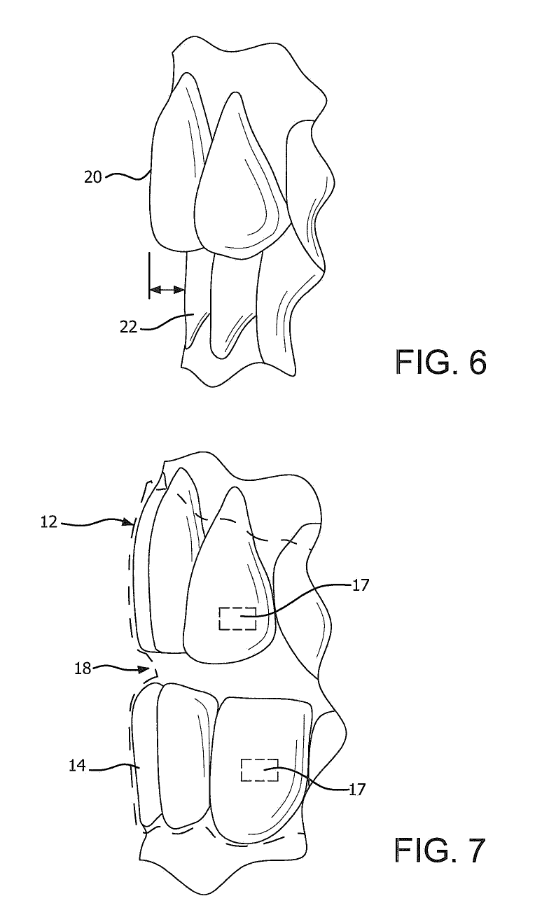

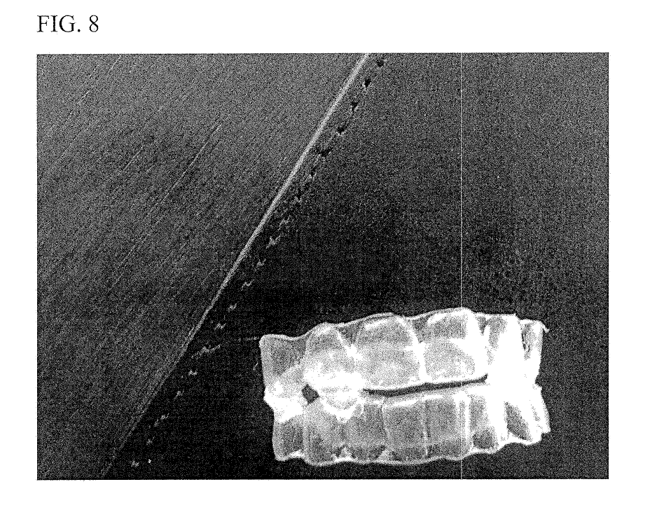

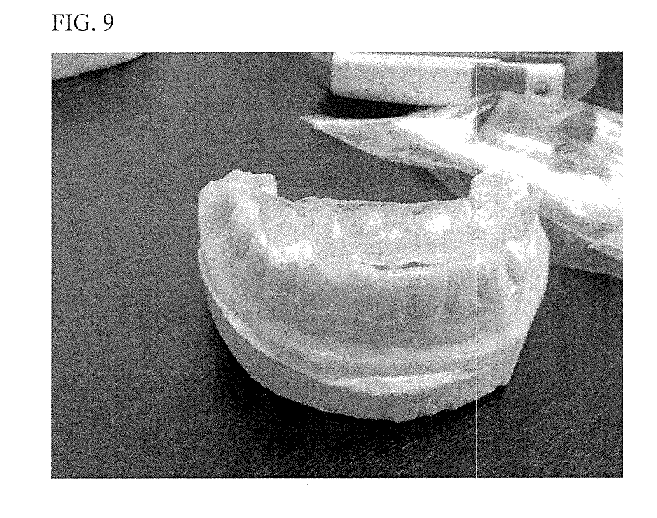

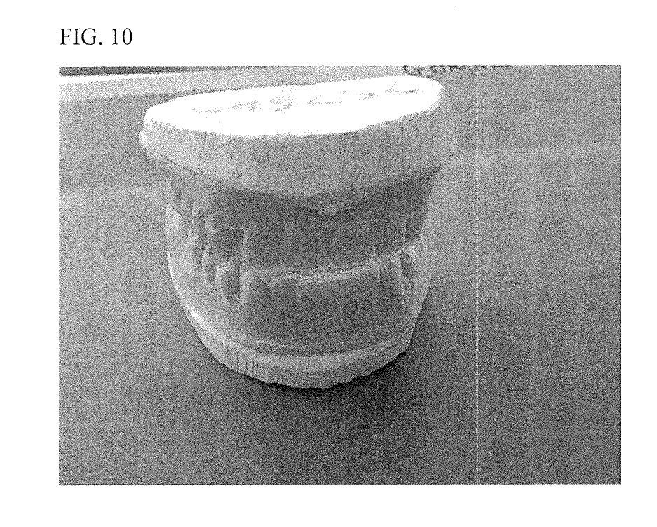

From a bite registration taken from the patient that delineates the normal bite position between the two jaws (described as the centric occlusion position) (See FIGS. 4 and 6). In the normal bite position, the front upper teeth 20 extend beyond the lower teeth 22.

The upper and lower receptors 12 and 14 of the device are placed together and the lower receptor 14 can advanced an incremental amount, initially about 2-3 mm as shown in FIGS. 5 and 7.

The receptors 12 and 14 are thus generally in line with each other thereby positioning the lower teeth 22 generally in line with the upper teeth 20 (FIG. 7), rather than the upper and lower teeth being offset (FIG. 6).

After cooling, the upper receptor 12 and lower receptor 14 are removed from the models and trimmed so that the lower (mandibular) receptor 14 extends preferably from the left first bicuspid to the right first bicuspid. It is possible to extend it over additional teeth.

The two receptors (upper and lower) 12 and 14 are then bonded together using a connecting structure 16 at spaced locations. The connecting structure 16 is preferably an adhesive. Any FDA approved bonding adhesive can be used, such as “BOND ALIGNER®” from Reliance Orthodontic Product, Inc. Preferably a clear aligner adhesive for thermoplastic aligners is used. Other adhesives that can be used are cyanoacrylate, acrylic adhesives, urethane adhesives and epoxies. The lower receptor 14 is offset from upper receptor 12 with upper receptor 12 being slightly in advance of lower receptor 12.

The now completed device 10 can be polished to remove any sharp or uneven surface and ready for patient use.

The device 10 can be used for treatment of sleep disorders which comprises a user inserting the device 10 produced according to the user's dentition in the user's mouth prior to sleeping.

This device 10 is custom made by dental professionals and will/can be modified by the dental professional as to the amount of advancement of the lower (mandibular) components to create the desired airway opening to produce the desired effect on snoring and sleep apnea. The amount of lower jaw advancement is determined by the dental professional.

Ideally, there would be several devices such as but not limited to two, three, four, five or six etc. made with incremental advancement, preferably about 1 to 3 mm of advancement of each of the lower receptor 14 is offset from the upper receptor 12 with the upper receptor 12 is slightly advance of the lower receptor 14 so that the user/patient can choose the device that fits most comfortably.

Another possible way to attach the device 10 to the teeth is to place a small amount of adhesive 17 on at least one of patient's teeth 20, 22. The adhesive 17 would be a light curing, radiopaque, nano-hybrid composite used for tooth colored fillings so that the shade of the adhesive matches the patients tooth.

If needed for additional customization of the device as requested by the dentist that requires additional retention on teeth to hold retainer in place can be accomplished by bonding small composite dots onto any tooth 22, 24.

The retentive dots are placed on the teeth 22, 24 prior to the impression or scan is taken of the teeth so that the retentive dot can be incorporated into the device. The adhesive can be put on two or four teeth. This will help the device 10 to stay on the teeth 20, 22. Placing adhesive 17 on teeth 20, 22 could be used instead or in addition to adhesive 16.

The key to the successful outcome of the device is to keep it as small as possible, but no smaller than the preferred size (upper canine to canine and lower first bicuspid to first bicuspid.)

All the references described above are incorporated by reference in their entirety for all useful purposes.

All materials used in the device or appliance are U.S. Food and Drug Administration (FDA) approved for intra oral use and therefore, the appliance will be able to also be FDA approved.

I claim:

1. A device for positioning the teeth for the treatment of sleeping disorders including snoring and obstructive sleep apnea (OSA) and OSA Syndrome, comprising an upper receptor capable of covering a partial amount of the user's upper teeth and lower receptor capable of covering a partial amount of the user's lower teeth and at least two connecting structures to connect the upper receptor to the lower receptor whereby a slot if formed between said upper receptor and said lower receptor to allow for breathing wherein said upper receptor and said lower receptor are generally aligned with each other to generally align the upper teeth with the lower teeth.

2. The device as claimed in claim 1, wherein said upper receptor and said lower receptor are made of identical medically-approved polymeric material.

3. The device as claimed in claim 1, wherein said upper receptor extends from the second bicuspid tooth to the second bicuspid tooth and said lower receptor extends the left second bicuspid tooth to the right second bicuspid tooth.

4. The device as claimed in claim 1, wherein said upper receptor extends from the right canine tooth to the left canine tooth and said lower receptor covers the left first bicuspid to the right first bicuspid.

5. The device as claimed in claim 2, wherein said upper receptor extends from the right canine tooth to the left canine tooth and said lower receptor covers the left first bicuspid to the right first bicuspid.

6. The device as claimed in claim 1 wherein said upper receptor and said lower receptor is at least about 1 mm thick.

7. The device as claimed in claim 1, wherein there are at least 3 connecting structures.

8. The device as claimed in claim 1, wherein there are at least 4 connecting structures with two connect structures connecting at each of the ends of the mold and three spaces between the upper and lower receptor.

9. The device as claimed in claim 4, wherein there are at least 4 connecting structures with two connect structures connecting at each of the ends of the mold and three spaces between the upper and lower receptor.

10. The device as claimed in claim 1, wherein the lower receptor is slightly offset from the normal bite position and lines up with the upper receptor.

11. The device as claimed in claim 8, wherein the lower receptor is slightly offset from the normal bite position and lines up with the upper receptor.

12. The device as claimed in claim 1, wherein the connecting structure is an adhesive.

13. The device as claimed in claim 10, wherein the connecting structure is an adhesive.

14. A method of fabricating the device as claimed in claim 1, which comprises
making an exact replica models of the patient's dentition and creating a model, wherein the model extends at most from the second bicuspid tooth to the second bicuspid tooth for the upper teeth and extends at most from the left second bicuspid tooth to the right second bicuspid tooth on the lower teeth,
thermoforming a plastic on the model to form an upper and lower receptor,
cooling the upper and lower receptor,
removing the upper and lower receptor from the model,
trimming that the lower and upper receptor and
bonding the upper and lower receptors together in at least two locations and thereby forming a slot in front between the two receptors and wherein the lower receptor is offset from upper receptor with upper receptor being slightly in advance of lower receptor.

15. A method of fabricating the device as claimed in claim 12, which comprises
making an exact replica models of the patients dentition and creating a model,
thermoforming a plastic on the model to form an upper and lower receptor,
cooling the upper and lower receptor,
removing the upper and lower receptor from the model,
trimming that the lower and upper receptor and
bonding the upper and lower receptors together in at least 2 locations and thereby faulting at least one slot in front between the two receptors and the lower receptor is offset from upper receptor with upper receptor being slightly in advance of lower receptor.

16. A treatment for sleep disorders which comprises a user inserting the device as claimed in claim 1 in the user's mouth prior to sleeping.

17. A treatment for sleep disorders which comprises a user inserting the device produced according to the method as claimed in claim 13 in the user's mouth prior to sleeping.

18. The treatment as claimed in claim 15, wherein at least two devices are made with incremental advancement of each of the lower receptors so that the upper receptor is slightly advanced of the lower receptor and the user selects the device that is the most comfortable to the user.

19. The treatment as claimed in claim 16, wherein the sleep disorder is snoring or obstructive sleep apnea.

20. A treatment for sleep disorders which comprises a user having an adhesive placed on at least on the user's teeth and inserting the device as claimed in claim 1 in the user's mouth prior to sleeping wherein the device covers the adhesive on the tooth.
